(19) United States
(12) Patent Application Publication
(10) Pub. No.: US 2025/0017554 A1
(43) Pub. Date: Jan. 16, 2025

(54) MEDICAL IMAGE PROCESSING APPARATUS, MEDICAL IMAGE PROCESSING METHOD, AND ULTRASONIC DIAGNOSIS APPARATUS

(71) Applicant: Canon Medical Systems Corporation, Otawara-shi (JP)

(72) Inventors: Keisuke MIYAKI, Utsunomiya (JP); Yasunori HONJO, Utsunomiya (JP)

(73) Assignee: Canon Medical Systems Corporation, Otawara-shi (JP)

(21) Appl. No.: 18/766,962

(22) Filed: Jul. 9, 2024

(30) Foreign Application Priority Data
Jul. 11, 2023 (JP) 2023-113693

(51) Int. Cl. A61B 8/08; A61B 8/00

(52) U.S. Cl. CPC A61B 8/0833; A61B 8/0883; A61B 8/463; A61B 8/483

(57) ABSTRACT

A medical image processing apparatus according to an embodiment includes processing circuitry. The processing circuitry acquires a medical image relating to a target part in which a device is implanted. The processing circuitry evaluates an implantation state of the device with respect to the target part from multiple angles, using the medical image. The processing circuitry outputs information based on the evaluation result of the implantation state.

### CROSS-REFERENCE TO RELATED APPLICATION(S)

This application is based upon and claims the benefit of priority from Japanese Patent Application No. 2023-113693, filed Jul. 11, 2023, the entire contents of which are incorporated herein by reference.

### FIELD

Embodiments described herein relate generally to a medical image processing apparatus, a medical image processing method, and an ultrasonic diagnosis apparatus.

### BACKGROUND

Transcatheter left atrial appendage closure using a left atrial appendage closure device is one of the most common treatment methods for reducing the risk of stroke due to atrial fibrillation. Transcatheter left atrial appendage closure is a treatment method in which a left atrial appendage closure device is implanted at the entrance of a left atrial appendage using a catheter, and it is necessary to check during the procedure whether the left atrial appendage closure device has been successfully implanted by evaluating the degree of deformation of the device after implantation. To check if the device has been implanted in the correct position, the device is pulled by a catheter after implantation, or the degree of compression of the device in a default four cross-sectional images is calculated. However, the method of pulling the left atrial appendage closure device only qualitatively evaluates the fixation, and the measurement in the default four cross-sectional images is based on a two-dimensional image, so there is a risk of overlooking a section that does not have a normal compression ratio.

### BRIEF DESCRIPTION OF THE DRAWINGS

FIG. 1 is a diagram showing a configuration of an ultrasonic diagnosis apparatus according to an embodiment.

FIG. 2 is a diagram schematically showing a left atrial appendage.

FIG. 3 is a diagram showing a technique of implanting a left atrial appendage closure device in a left atrial appendage.

FIG. 4 is a side-view diagram of the left atrial appendage closure device.

FIG. 5 is a plan view of the left atrial appendage closure device.

FIG. 6 is a diagram showing a diameter [mm] of an entrance of a left atrial appendage and an implantable range [mm] for the left atrial appendage closure device.

FIG. 7 is a diagram illustrating a procedure of an implantation state evaluation process for the left atrium appendage closure device by the ultrasonic diagnosis apparatus.

FIG. 8 is a diagram schematically showing a three-dimensional ultrasonic image.

FIG. 9 is a diagram illustrating an axis core orthogonal cross-sectional image and an axis core parallel cross-sectional image.

FIG. 10 is a diagram illustrating a device diameter map showing a measured value and proper values of each line segment angle.

FIG. 11 is a diagram illustrating a device diameter graph showing a measured value and proper values of each line segment angle.

FIG. 12 is a diagram showing an example of a display screen including a superimposed image of a device diameter map and an axis core orthogonal cross-sectional image.

FIG. 13 is a diagram showing a device diameter map showing a determination result according to Modification 2.

FIG. 14 is a diagram illustrating a device diameter map showing measured values of dimensions before and after a tensile test.

FIG. 15 is a diagram illustrating a device diameter graph showing measured values of dimensions before and after a tensile test.

FIG. 16 is a diagram illustrating an electrocardiogram and a device diameter map.

FIG. 17 is a diagram showing a configuration example of a medical image processing apparatus according to Modification 6.

### DETAILED DESCRIPTION

A medical image processing apparatus according to an embodiment has an acquisition unit, an evaluation unit, and an output control unit. The acquisition unit acquires a medical image relating to a target part at which a device is implanted. The evaluation unit evaluates an implantation state of the device in relation to the target part from a plurality of angles, using the medical image. The output control unit outputs information based on a result of evaluation of the implantation state.

In the following description, the medical image processing apparatus, a medical image processing program, and an ultrasonic diagnosis apparatus according to the embodiment will be explained with reference to the drawings.

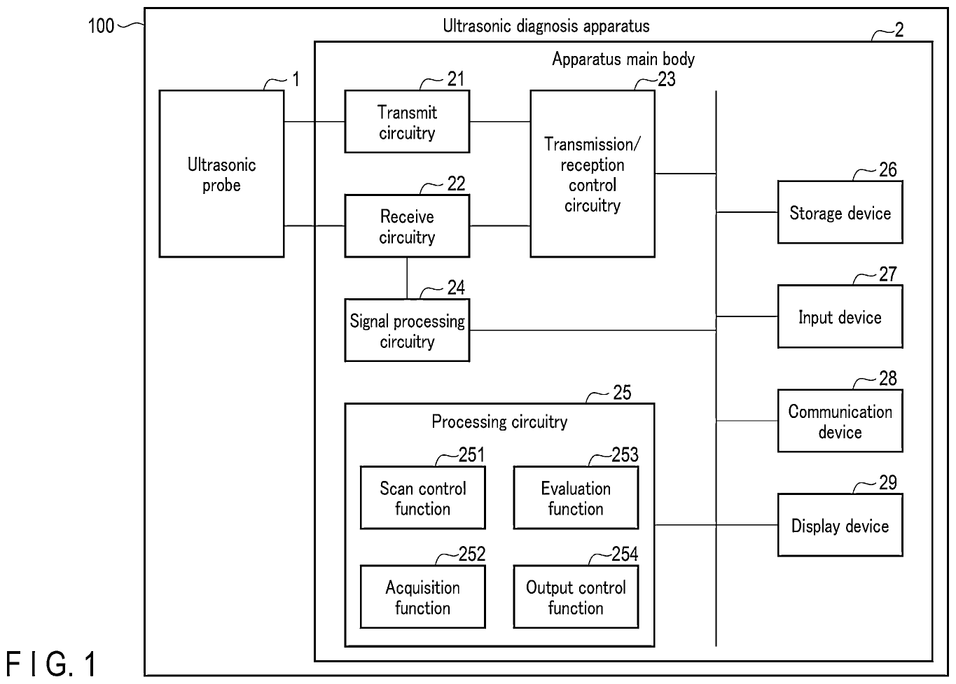

FIG. 1 is a diagram showing a configuration example of an ultrasonic diagnosis apparatus 100 according to the embodiment. As shown in FIG. 1, the ultrasonic diagnosis apparatus 100 has an ultrasonic probe 1 and an apparatus main body 2. The apparatus main body 2 is a computer provided with transmit circuitry 21, receive circuitry 22, transmission/reception control circuitry 23, signal processing circuitry 24, processing circuitry 25, a storage apparatus 26, an input device 27, a communication device 28, and a display device 29. The apparatus main body 2 is an example of a medical image processing apparatus. The ultrasonic probe 1 is connected to the apparatus main body 2 in a detachable manner.

The ultrasonic probe 1 is a probe that has a role of receiving and transmitting ultrasonic waves. The ultrasonic probe 1 has a plurality of vibrators that are one-dimensionally or two-dimensionally arranged. Each vibrator transmits an ultrasonic wave in response to a drive signal supplied from the transmit circuitry 21. A delay time is imparted to each signal to converge an ultrasonic wave transmitted from the entire ultrasonic probe 1 into a beam shape. The generated ultrasonic wave is reflected on a non-matching surface of an acoustic impedance in a subject's body. Each vibrator converts the ultrasonic wave reflected within the subject's body (reflected wave) into an echo signal. The ultrasonic probe 1 may be a linear probe, a convex probe, a sector probe, a radial probe, an arc probe, a circular probe, or any other probe having any arrangement.

The transmit circuitry 21 transmits a beam-shaped ultrasonic wave via the ultrasonic probe 1 in compliance with the control of the transmission/reception control circuitry 23. Specifically, the transmit circuitry 21 imparts a delay time for giving transmission directivity to each drive signal and supplies the drive signal to each vibrator, so that a beam-shaped ultrasonic wave deflected at a given transmit beam angle is transmitted. The transmit circuitry 21 repeats an ultrasonic wave transmission, changing the transmit beam angle.

The receive circuitry 22 receives via the ultrasonic probe 1 a receive signal corresponding to an ultrasonic wave transmission transmitted by the transmit circuitry 21 under the control from the transmission/reception control circuitry 23. Specifically, the receive circuitry 22 imparts a focusing delay time for giving a reflected wave and a deflection delay time for giving a reception directivity to each of the echo signals received by the ultrasonic probe 1, performs phased addition on these echo signals, and generates a receive signal corresponding to a received beam. The generation of a receive signal may be called “beam forming”.

The transmission/reception control circuitry 23 synchronously controls the transmit circuitry 21 and the receive circuitry 22, so that three-dimensional ultrasonic scanning is performed on an imaging target part in the subject's body via the ultrasonic probe 1.

The signal processing circuitry 24 performs B-mode processing and color Doppler processing, etc. on a received signal from the receive circuitry 22. With the B-mode processing, the signal processing circuitry 24 performs logarithmic amplification, envelope detection processing, and logarithmic compression on a received signal from the receive circuitry 22, and generates B-mode information in which a signal strength is expressed in a brightness level of luminance. The signal processing circuitry 24 generates a two-dimensional or a three-dimensional B-mode image in which a signal strength is expressed in a luminance value based on B-mode information. In the Doppler processing, the signal processing circuitry 24 conducts a frequency analysis on a receive signal from the receive circuitry 22, and estimates Doppler information, such as a speed, dispersion, and power of a moving object, such as blood and tissue, for each sample point. The signal processing circuitry 24 subsequently generates a two-dimensional or three-dimensional Doppler image in which a speed, dispersion, and power of a moving object, such as blood and tissue, are expressed in color values. If a B-mode image and a Doppler image are not distinguished, these images may be called an “ultrasonic image”. The ultrasonic images are stored in the storage apparatus 26. The signal processing circuitry 24 can be realized by a discretionarily selected processor.

The processing circuitry 25 has a processor such as a central processing unit (CPU), etc., that governs the ultrasonic diagnosis apparatus 100. The processing circuitry 25 executes an ultrasonic diagnosis program stored in the storage apparatus 26 to realize a function corresponding to the program. The processing circuitry 25 realizes the scan control function 251, the acquisition function 252, the evaluation function 253, and the output control function 254, for example. The embodiment is not limited to the case in which the respective functions 251 to 254 are realized by single processing circuitry. The functions 251 to 254 may be realized by composing processing circuitry by combining a plurality of independent processors, and executing the respective processor control programs. Each of the functions 251 to 254 may be implemented as a module constituting a control program or as separate hardware.

The processing circuitry 25 controls, through realization of the scan control function 251, the transmission/reception control circuitry 23 and the signal processing circuitry 24 for performing three-dimensional ultrasonic scanning on an imaging target part in the subject's body. The signal processing circuitry 24 generates an ultrasonic image through performance of three-dimensional ultrasonic wave scanning, as described above.

By the realization of the acquisition function 252, the processing circuitry 25 acquires various information items. As an example, the processing circuitry 25 acquires a medical image relating to a target part at which a device is implanted. As a medical image, a three-dimensional ultrasonic image generated by the signal processing circuitry 24 is typically acquired. The device is implanted in a target part within the subject's body. More specifically, it suffices that the device is a device that can invade a target part while being in a compressed state and can be adhered to the target part by being uncompressed. Examples of a device according to the present embodiment include a left atrial appendage closure device, a balloon, a stent, etc.

The processing circuitry 25 evaluates, through realization of the evaluation function 253, the implantation state of the device in relation to the target part from multiple angles, using a medical image acquired through the acquisition function 252. Typically, the processing circuitry 25 evaluates the implantation state from a plurality of angles around the axis core of the device. The implantation state can be evaluated by the suitability of a device for implantation in a target part, dimensions of a device implanted in a target part in a predetermined cross-section, and the like. As an example, the processing circuitry 25 measures, from each angle, dimensions of the device implanted in the target part based on a three-dimensional ultrasonic image.

The processing circuitry 25 outputs various information through realization of the output control function 254. The output destination may be a storage apparatus 26 and a display device 29 or a computer connected via the communication device 28. As an example, the processing circuitry 25 causes the display device 29 to display information based on a result of the evaluation of the implantation state obtained by the evaluation function 253, and specifically dimensions of the device implanted in the target part at multiple angles. Alternatively, the processing circuitry 25 may display a measured value and a proper value of the dimensions at multiple angles on a map or a graph.

The storage apparatus 26 is a type of storage apparatus that stores various types of information, such as a hard disk drive (HDD), a solid state drive (SSD), or an integrated circuit storage device, etc. The storage apparatus 26 may also be, for example, a drive that performs reading and writing of various kinds of information on a portable storage medium, such as a CD-ROM drive, a DVD drive, or a flash memory. For example, the storage apparatus 26 stores an ultrasonic diagnosis program, etc. of the ultrasonic diagnosis apparatus 100.

The input device 27 is various types of user interfaces on a touch panel or an operation panel. An operator can input various operations and commands into the ultrasonic diagnosis apparatus 100 via the input device 27. The input device 27 may be a speech recognition device that converts audio signals collected by a microphone into command signals.

The communication device 28 is an interface performing data communications with a picture archiving and communication system (PACS) server, a hospital information system (HIS) server, a modality worklist management (MWM) server, or the like, via a local area network (LAN) or the like.

The display device 29 displays various types of information in response to a command from the processing circuitry 25. As the display device 29, for example, a liquid crystal display (LCD), a cathode ray tube (CRT) display, an organic electro luminescence display (OELD), a plasma display, or any other display can be used as appropriate. A projector may be provided as the display device 29.

Hereinafter, an example of the operation of the ultrasonic diagnosis apparatus 100 according to the present embodiment will be explained in detail.

The ultrasonic diagnosis apparatus 100 is provided for a technique of implanting a device in a target site. Although not limited to a particular technique, the technique according to the present embodiment is a left atrial appendage closure technique, for example. The left atrial appendage closure technique is a less invasive treatment method with which a left atrial appendage closure device is implanted in a left atrial appendage (LAA). Herein, the left atrial appendage is an example of a “target part”, and the left atrial appendage closure device is an example of a “device”. The left atrial appendage closure device is not limited to a particular one as long as it is a device to be implanted in a left atrial appendage, and a Watchman (registered trademark) device (Boston Scientific) or an improved version of a Watchman (registered trademark) device is assumed.

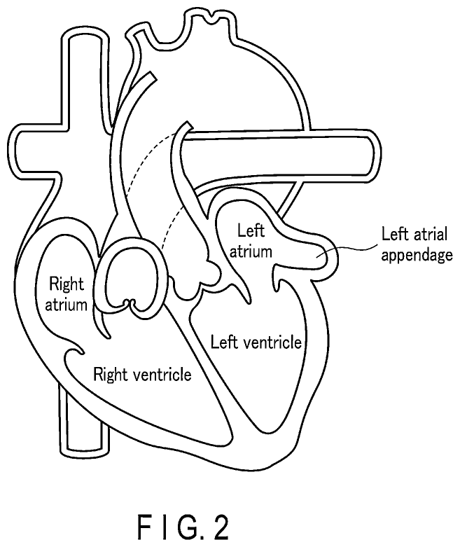

FIG. 2 is a diagram schematically showing a left atrial appendage. As shown in FIG. 2, the heart has a right atrium, a right ventricle, a left atrium, and a left ventricle. There is a small sac-shaped space called a left atrial appendage in the left atrium. If atrial fibrillation causes blood to linger in an atrium, a mass of blood (thrombus) is easily formed in a left atrial appendage. If the thrombi dislodged from the left atrial appendage reach a brain through an artery and block a brain blood vessel, a stroke is caused. A risk of stroke can be reduced by occluding a left atrial appendage.

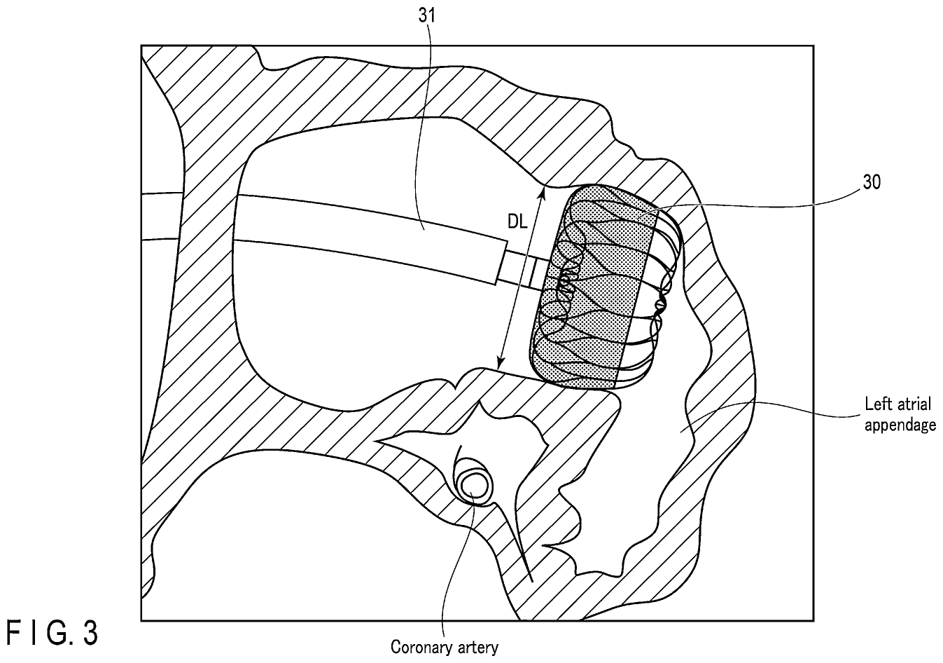

FIG. 3 is a diagram showing a technique of implanting the left atrial appendage closure device 30 in a left atrial appendage. The device 30 is detachably connected to the catheter 31. The operator inserts the catheter 31 through the vein at the base of a leg, and guides the device 30 to the left atrial appendage. Thereafter, the operator deploys the device 30 and tightly adheres the device 30 against the inner wall of the left atrial appendage. After the adhesion is ensured, the operator disengages the device 30 from the catheter 31 to leave behind the left atrial appendage closure device 30 permanently. After the left atrial appendage closure device 30 is left in the left atrial appendage, the device 30 will be slowly covered by the tunica intima of the left atrial appendage and will eventually close the left atrial appendage completely. Leaving the left atrial appendage closure device 30 in the left atrial appendage can prevent a flow of thrombi caused in the left atrial appendage into the outside thereof and keep the thrombi inside the left atrial appendage. This can reduce the risk of stroke.

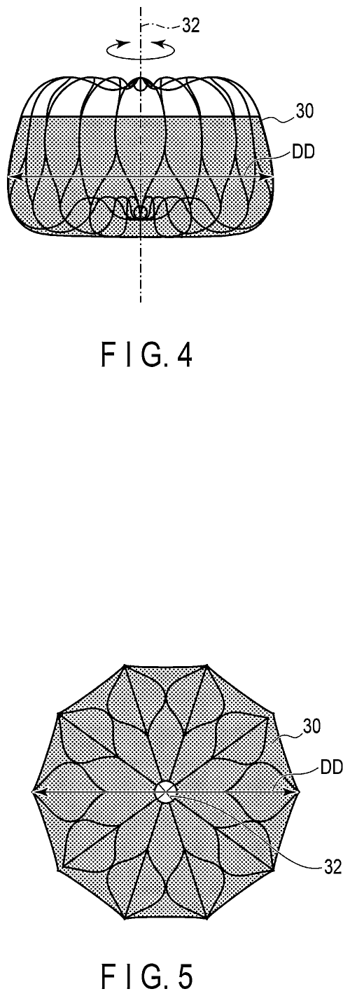

FIG. 4 is a side view of the left atrial appendage closure device 30, and FIG. 5 is a plan view of the same. As shown in FIGS. 4 and 5, the left atrial appendage closure device has a metal-made skeleton and a net covering the skeleton that can together be deployed around the axis core 32. After the left atrial appendage closure device 30 is left in the left atrial appendage, the net will be slowly endothelialized by the tunica intima of the left atrial appendage. The dimension of the left atrial appendage closure device 30 in a direction orthogonal to the axis core 32 will be called a “device diameter”. A maximum device diameter will be called a “maximum diameter DD”. The maximum diameter when the left atrial appendage closure device 30 is deployed to its limit under a circumstance where no pressure from the left atrial appendage is applied to the device will be called a “device size”. The device size is determined according to the entrance diameter DL of the left atrial appendage of a subject (see FIG. 3).

The entrance is measured by intraoperative transesophageal echocardiography (IOTEE) before the implantation of the left atrial appendage closure device. It is preferable if IOTEE is performed by the scan control function 251 of the processing circuitry 25. Specifically, the entrance diameter of the left atrial appendage is measured from four two-dimensional scanning surfaces respectively corresponding to four probe angles before the left atrial appendage closure device is implanted. The probe angle means an angle around the axis core of the ultrasonic probe 1. The probe angle of 0 degrees can be discretionarily set. The probe angle is not limited to a particular angle, but it is recommended to set the probe angle at intervals of 45 degrees, such as 0°, 45°, 90°, and 135°.

A plurality of left atrial appendage closure devices are prepared for respectively corresponding device sizes. If the device size of the left atrial appendage closure device is too large for the entrance diameter of the left atrial appendage, there is a risk of pressing the coronary artery by the left atrial appendage closure device; if the device size is too small, there is a risk of deviation of the left atrial appendage closure device from the left atrial appendage. For this reason, the left atrial appendage to be used is determined in accordance with an entrance diameter of the left atrial appendage measured by IOTEE.

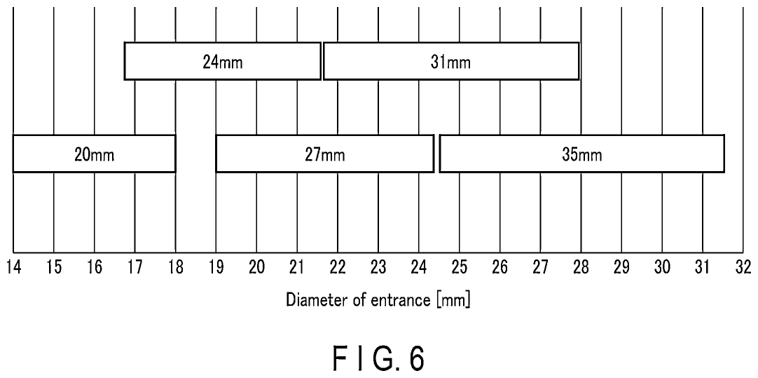

FIG. 6 is a diagram showing a diameter [mm] of an entrance of a left atrial appendage and an implantable range [mm] for the left atrial appendage closure device. The horizontal axis of the graph of FIG. 6 represents an entrance diameter of the left atrial appendage, and the device size of the left atrial appendage closure device that is applicable to each entrance diameter is superimposed thereon. For example, a left atrial appendage closure device having a device size of 31 mm can be applied to the left atrial appendage having an entrance diameter in the range from 21.5 mm to 28 mm. As another example, a left atrial appendage closure device having a device size of 24 mm or 27 mm is applicable to the left atrial appendage having an entrance diameter of 20 mm.

The adhesion of the left atrial appendage closure device to the left atrial appendage is checked by a tensile test of the left atrial appendage closure device called a “tag test”. In four two-dimensional scanning surfaces similar to those in IOTEE performed before implantation of the left atrial appendage closure device, a maximum diameter of the left atrial appendage closure device adhered to the left atrial appendage is measured, and the compression ratio, which is a ratio of the device size to the measured value, is measured. If the compression ratio falls under the range between 10% and 30%, it is determined that the device is normally implanted. For example, it is said that the proper value of the device size falls between 21 mm and 27 mm.

However, the method of pulling the left atrial appendage closure device is no more than a qualitative evaluation of the degree of adhesion, and the method may lack accuracy. Although the compression ratio is measured with four two-dimensional scanning cross sections, there is a possibility that these cross sections may not be appropriate for measuring the compression rate, since the measurement would be performed on a two-dimensional scanning surface. It is difficult to accurately evaluate a diameter of the left atrial appendage closure device on a two-dimensional scanning surface to begin with.

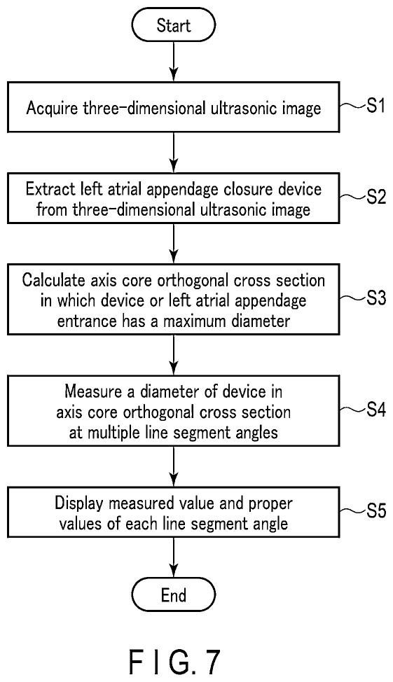

Hereinafter, an example of the operation of the implantation state evaluation processing by the ultrasonic diagnosis apparatus 100 according to the present embodiment for the left atrial appendage closure device is explained, with reference to FIG. 7. FIG. 7 is a diagram illustrating a procedure of an implantation state evaluation process for a left atrium appendage closure device by an ultrasonic diagnosis apparatus 100. Suppose the left atrial appendage closure device is implanted in a left atrial appendage of a subject at the starting time of step S1 in FIG. 7. A catheter is not necessarily disengaged from the left atrial appendage closure device.

First, the processing circuitry 25 acquires a three-dimensional ultrasonic image through realization of the acquisition function 252 (step S1). Assume a three-dimensional B-mode image as a three-dimensional ultrasonic image. Specifically, with the ultrasonic probe 1 abutted to the chest of the subject, three-dimensional ultrasonic scanning is performed on the heart including the left atrial appendage as an imaging target part under the control of the scan control function 251. The signal processing circuitry 24 generates a three-dimensional ultrasonic image, which is a three-dimensional B-mode image based on a receive signal acquired from the receive circuitry 22 through three-dimensional ultrasonic scanning. The left atrial appendage of the subject and the left atrial appendage closure device implanted in the left atrial appendage are shown on the three-dimensional ultrasonic image. A three-dimensional ultrasonic image is stored in the storage apparatus 26.

After step S1, the processing circuitry 25 extracts, through realization of the evaluation function 253, a left atrial appendage closure device from the three-dimensional ultrasonic image acquired in step S1 (step S2). Specifically, in step S2, the processing circuitry 25 performs image processing on the three-dimensional ultrasonic image, and extracts an image region corresponding to the left atrial appendage closure device from the three-dimensional ultrasonic image. As the image processing, extraction using empirical rules, such as a luminance value of the left atrial appendage closure device and a shape of the device in the three-dimensional ultrasonic image, can be used as appropriate. Machine learning in which a three-dimensional ultrasonic image is input and a location of the left atrial appendage closure device is output using a neural network may be used as the image processing.

After step S2, the processing circuitry 25 calculates, through realization of the evaluation function 253, an axis core orthogonal cross section in which the left atrial appendage closure device or the left atrial appendage entrance takes a maximum diameter (step S3). Hereinafter, it is assumed that the processing circuitry 25 calculates an axis core orthogonal cross section in which the left atrial appendage closure device takes a maximum diameter. The axis core orthogonal cross section is a cross section that is orthogonal to the axis core of the left atrial appendage closure device. The axis core orthogonal cross section in which the left atrial appendage entrance takes a maximum diameter means an axis core orthogonal cross section in which the entrance diameter of the left atrial appendage is a maximum value. The processing hereinafter can be performed for the case of the axis core orthogonal cross section in which the left atrial appendage entrance takes a maximum diameter, similarly to the case of the axis core orthogonal cross section in which the left atrial appendage closure device takes a maximum diameter.

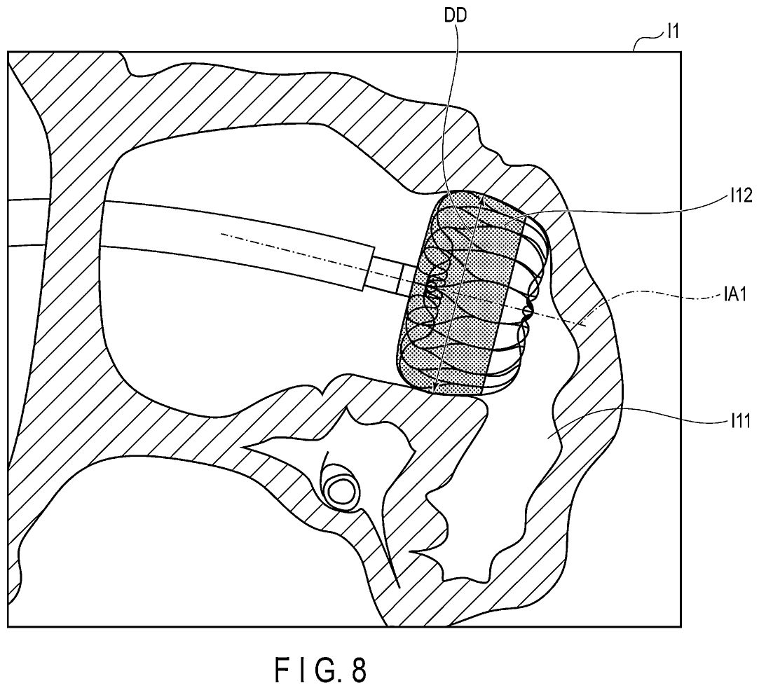

FIG. 8 schematically shows the three-dimensional ultrasonic image I1. As shown in FIG. 8, the left atrial appendage I11 is shown on the three-dimensional ultrasonic image I1. The left atrial appendage closure device I12 is implanted in the left atrial appendage I11. The left atrial appendage closure device I12 is extracted in step S2. The processing circuitry 25 calculates the axis core IA1 of the left atrial appendage closure device I12 based on a luminance value distribution and a shape, etc. of the left atrial appendage closure device I12. The processing circuitry 25 measures the dimension (device diameter) of the left atrial appendage closure device I12 along the axis core IA1 at predetermined intervals, and specifies a maximum diameter DD. The processing circuitry 25 calculates the surface that is orthogonal to the axis core IA1 and includes the maximum diameter DD as an axis core orthogonal cross section that takes a maximum diameter. The processing circuitry 25 may calculate other cross sections having the axis core IA1 as a point of reference. For example, the processing circuitry 25 may calculate a surface that is parallel to the axis core IA1 and includes the maximum diameter DD (the axis core parallel cross section). The processing circuitry 25 performs multi-planar reconstruction (MPR) processing on the three-dimensional ultrasonic image, and generates an MPR cross-sectional image relating to the axis core orthogonal cross section (axis core orthogonal cross-sectional image) and an MPR cross-sectional image relating to the axis core parallel cross section (axis core parallel cross-sectional image).

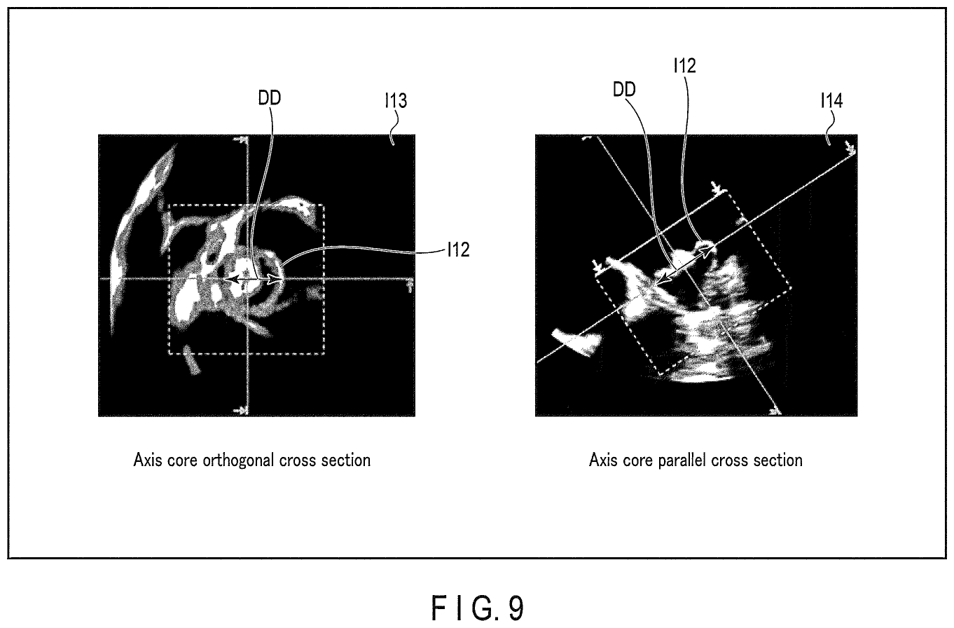

FIG. 9 is a diagram illustrating an axis core orthogonal cross-sectional image I13 and an axis core parallel cross-sectional image I14. As shown in FIG. 9, the axis core orthogonal cross-sectional image I13 is an image of a cross section orthogonal to the axis core of the left atrial appendage closure device, and the left atrial appendage closure device I12 is shown therein in a ring shape. In the axis core orthogonal cross-sectional image I13, a change in the device diameter of the left atrial appendage closure device I12 around the axis core can be observed. The axis core parallel cross-sectional image I14 is an image of a cross section parallel to the axis core of the left atrial appendage closure device I12. In the axis core parallel cross-sectional image I14, adhesion between the left atrial appendage closure device I12 and the left atrial appendage can be observed.

After step S3, the processing circuitry 25 measures, through realization of the evaluation function 253, a diameter of the left atrial appendage closure device in the axis core orthogonal cross section calculated in step S3 for multiple angles (step S4). In step S4, the processing circuitry 25 specifies a point on the axis core (hereinafter, an “axis core point”) of the left atrial appendage closure device in the axis core orthogonal cross section, and calculates a line (hereinafter, a “line segment angle”) that passes the axis core point and connects both ends of the left atrial appendage closure device for multiple angles around the axis core point. Suppose that the line segment angle is 0 degrees on the +X axis (horizontal axis) on the axis core orthogonal cross section, and increases as the line rotates in a counter-clockwise manner. The processing circuitry 25 measures the length of the line as a device diameter. The measured device diameter may be called a “measured value”.

After step S4, the processing circuitry 25 displays a measured value and a proper value of each line segment angle through realization of the output control function 254 (step S5). In step S5, the processing circuitry 25 causes the display device 29 to display the measured value and the proper value of each line segment angle in a predetermined layout. The measured value and the proper value of each line segment angle are displayed in a layout, such as a device diameter map and a device diameter graph, for example. The layout can be discretionarily selected in accordance with a user's instruction.

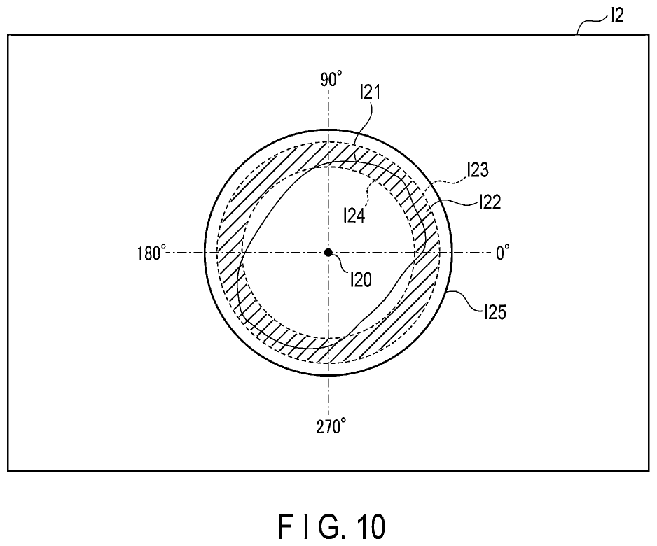

FIG. 10 is a diagram illustrating a device diameter map I2 showing the measured value I21 and the proper values I22, I23, and I24 of each line segment angle. As shown in FIG. 10, the device diameter map I2 is a schematic map of the axis core orthogonal cross section in which the measured value I21 and the proper values I22, I23, and I24 are plotted for each line segment angle around the axis core point I20.

As described above, the measured value I21 represents a distance between both ends of the left atrial appendage closure device passing the axis core point I20. In other words, the curve that the measured value I21 draws represents a contour of the left atrial appendage closure device in the axis core orthogonal cross section. It suffices that the proper range I22, the upper-limit proper value I23, and/or the lower-limit proper value I24 are drawn as the proper values. The proper range I22 is a range between the upper-limit proper value I23 and the lower-limit proper value I24. If the measured value I21 falls under the proper range I22, it means that the left atrial appendage closure device is properly adhered. The upper-limit proper value I23 and the lower-limit proper value I24 are determined based on a device size. As an example, if the proper range of the compression ratio of the device size is 10% to 30%, the upper-limit proper value I23 is the compression ratio 10% of the device size of the left atrial appendage closure device to be used, and the lower-limit proper value I24 is the compression ratio 30% of the device size of the left atrial appendage closure device to be used. The processing circuitry 25 may display the proper range I22 and the other range (improper range) with distinctions by colors. The processing circuitry 25 may draw the device size I25 on the device diameter map I2. The device size I25 may be designated through the input device 27, or may be stored in the storage apparatus 26 in advance in such a manner that the device size can be searchable.

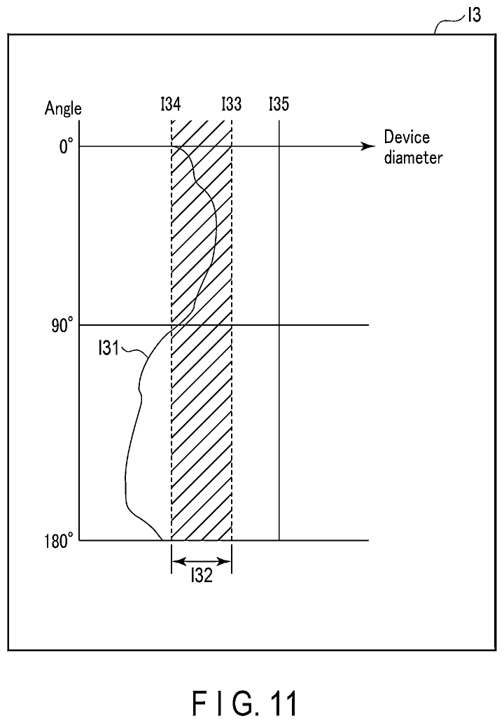

FIG. 11 is a diagram illustrating a device diameter graph I3 showing the measured value I31 and the proper values I32, I33, and I34 of each line segment angle. As shown in FIG. 11, the horizontal axis represents a device diameter and the vertical axis represents a line segment angle in the device diameter graph I3. In the device diameter graph I3, the measured value I31, the proper values I32, I33, and I34, and the device size I35 are plotted for each line segment angle around the axis core point. It suffices that the proper range I32, the upper-limit proper value I33, and/or the lower-limit proper value I34 are drawn as the proper values, similarly to the proper range I22, the upper-limit proper value I23, and/or the lower-limit proper value I24 of the device diameter map I2 shown in FIG. 10. The device diameter graph I3 is not limited to the graph shown in FIG. 11, and it may be a graph expressing the relationship between the device diameter and the line segment angle, such as a bar graph or a pie chart.

As described above, the measured value and the proper values of a device diameter (dimension) of the left atrial appendage closure device for each line segment angle are plotted in the device diameter map and a device diameter graph. A user can exhaustively check the relationship between the measured value and the proper values for multiple line segment angles by checking such a device diameter map and a device diameter graph. The user can also easily visually check whether or not the measured value falls under a proper range for each line segment angle. The user can check the relationship between the measured value and the proper values on the same coordinate system as the axis core orthogonal cross section by using a device diameter map. The user can clearly ascertain the distribution of the measured value and the proper values of each line segment angle by using a device diameter graph.

If the measured value does not fall under a proper range, it is estimated that the left atrial appendage closure device is not appropriately adhered to the left atrial appendage at the line segment angle at which the measured value is acquired; if the measured value falls under a proper range, on the other hand, it is estimated that the left atrial appendage closure device is appropriately adhered to the left atrial appendage at the line segment angle at which the measured value is acquired. The user can thus determine whether or not the left atrial appendage closure device is appropriately adhered to or implanted in the left atrial appendage for each line segment angle. If there is a line segment angle at which the left atrial appendage closure device is not appropriately adhered to the left atrial appendage, the user needs to determine whether or not the implantation of the left atrial appendage closure device should be re-done. If the left atrial appendage closure device is adhered to the left atrial appendage at all line segment angles, the user determines that the left atrial appendage closure device is appropriately implanted.

It suffices that the processing circuitry 25 displays a device diameter map or a device diameter graph that is selected via the input device 27. The processing circuitry 25 may display a device diameter map and a device diameter graph side by side.

After step S5, the implantation state evaluation processing on the left atrial appendage closure device is finished.

The foregoing embodiment is merely an example, and deletion, addition and/or changing of each element described in the above can be made to the present embodiment to the extent that the gist of the embodiment is not deviated from.

### Modification 1

The processing circuitry 25 according to Modification 1 displays an axis core orthogonal cross-sectional image of a cross-section orthogonal to the axis core of the device in the three-dimensional ultrasonic image, and displays the measured value and the proper values of the dimension for each line segment angle on the axis core orthogonal cross-sectional image. Specifically, the processing circuitry 25 superimposes the measured value, the proper values of the device diameter of the left atrial appendage closure device, and/or the device size for each line segment angle on the axis core orthogonal cross-sectional image, and displays the superimposed image.

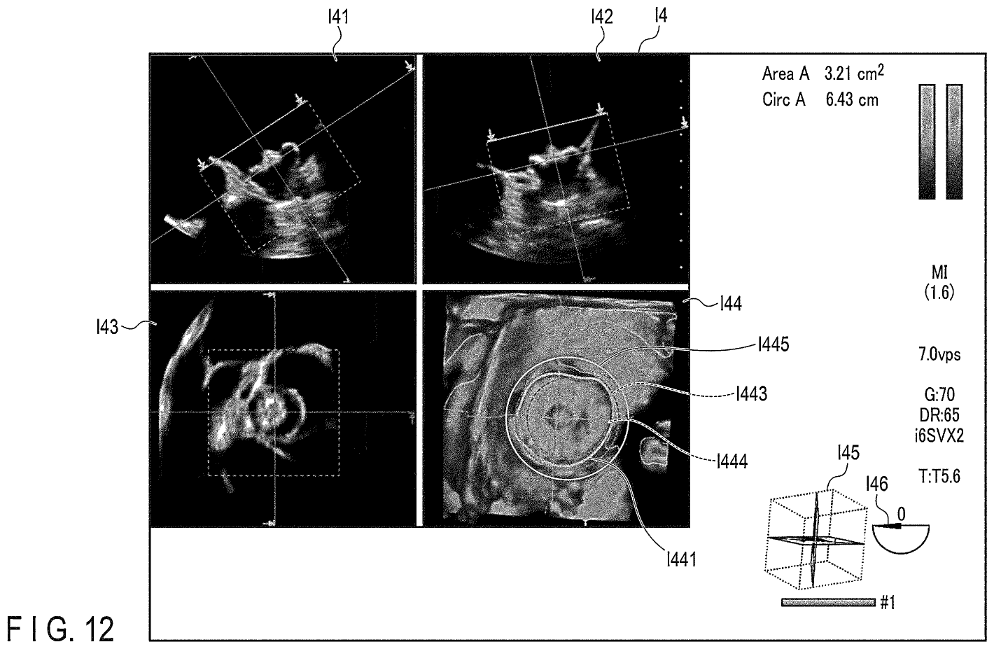

FIG. 12 is a diagram showing an example of a display screen I4 including a superimposed image I44 of a device diameter map and an axis core orthogonal cross-sectional image. As shown in FIG. 12, the processing circuitry 25 displays, in the display screen I4, the axis core parallel cross-sectional image I41, the axis core parallel cross-sectional image I42, the axis core orthogonal cross-sectional image I43, and the superimposed image I44, side by side. The axis core parallel cross-sectional image I41 is generated based on a three-dimensional ultrasonic image, and represents the cross section corresponding to the designated line segment angle. The axis core parallel cross-sectional image I41 shows a pseudo two-dimensional scanning cross-sectional image having the designated line segment angle as a probe angle. The axis core parallel cross-sectional image I42 is generated based on a three-dimensional ultrasonic image, and represents a cross section corresponding to an angle orthogonal to the designated line segment angle. The axis core parallel cross-sectional image I42 shows a pseudo two-dimensional scanning cross-sectional image having an angle orthogonal to the designated line segment angle as a probe angle. The axis core orthogonal cross-sectional image I43 is an axis core orthogonal cross-sectional image on which the device diameter map is not superimposed. A marker I45 indicating the positions of the cross sections of the cross-sectional images I41, I42, and I43 and a marker I46 indicating a probe angle may be further displayed in the display screen I4. The markers indicating the cross section positions of the cross-sectional images I41, I42, and I43 may be superimposed on each of the cross-sectional images I41, I42, and I43.

As shown in FIG. 12, in the superimposed image I44, the measured value I441, the proper values I442, I443, and I444, and the device size I445 are superimposed on the axis core orthogonal cross-sectional image. The curve indicating the measured value I441, the proper values I442, I443, and I444, and the device size I445 are displayed with distinctions for visibility by color values and line types. For example, it is desirable if the curve representing the measured value I441 is displayed as a blue solid line, the curve representing the upper-limit proper value I443 and the lower-limit proper value I444 is displayed as a red dotted line, and the curve representing the device size I445 is displayed as a yellow solid line.

As shown in Modification 1, displaying the superimposed image allows the user to check the measured value and the proper values of the diameter of the left atrial appendage closure device on the axis core orthogonal cross-sectional image. This allows a user to determine (in) appropriateness of adhesion or implantation of the left atrial appendage closure device in additional consideration of the position relationship between the device and the echo image of the left atrial appendage.

### Modification 2

The processing circuitry 25 according to Modification 2 determines whether or not the left atrial appendage closure device is appropriately implanted in the left atrial appendage for each line segment angle based on a measured value and proper values of the dimension. Specifically, the processing circuitry 25 determines that the measured value is proper if it falls under the proper range, and determines that the measured value is improper if it does not fall under the proper range. The processing circuitry 25 displays the determination result regarding the (in) appropriateness of the implantation of the left atrial appendage closure device for the dimension of each of the multiple line segment angles. The processing circuitry 25 causes the display device 29 to display a display screen of the determination result. As an example of the display of the determination result, the processing circuitry 25 displays the angle range in which the left atrial appendage closure device is appropriately implanted and the angle range in which the left atrial appendage closure device is inappropriately implanted by different visual effects. The determination result is displayed in a device diameter map and/or a device diameter graph.

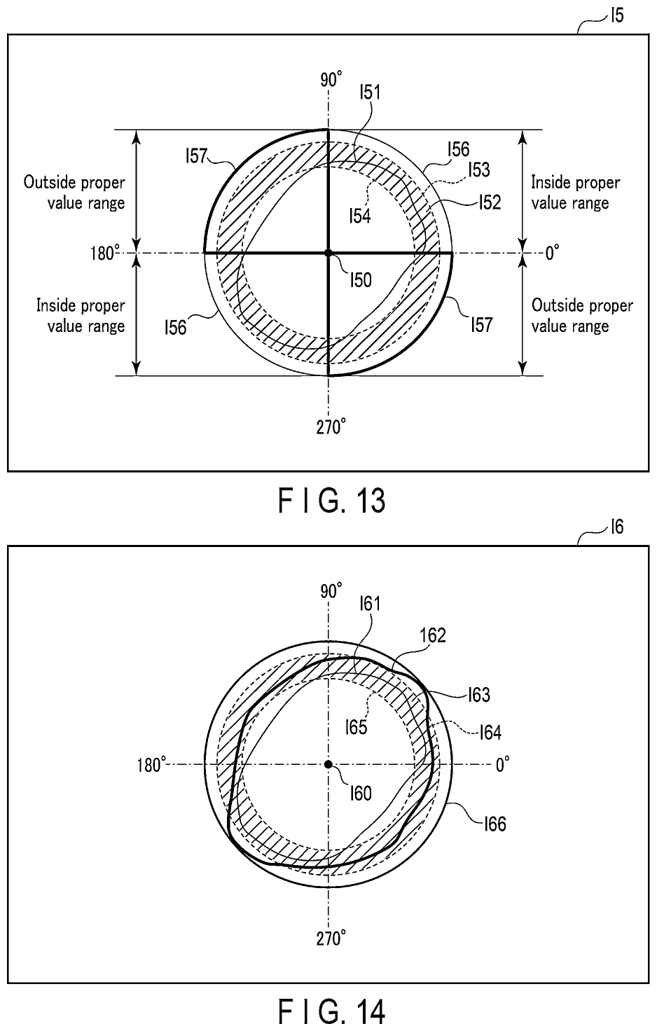

FIG. 13 is a diagram showing a device diameter map I5 showing a determination result according to Modification 2. As shown in FIG. 13, the measured value I51 and the proper values I52, 153, and I54 are drawn in the device diameter map I5 for each line segment angle around the axis core point I50. The markers I56 and I57 representing a determination result of (non-) appropriateness of the implantation of the left atrial appendage closure device is drawn on the device diameter map I5. Specifically, the line segment angle range where the measured value I51 falls under the proper range I52 is decorated by the appropriateness maker I56. In more detail, the periphery of the line segment range where the measured value I51 falls within the proper range I52 is fringed by the appropriateness marker I56. Similarly, the line segment range where the measured value I51 falls outside of the proper range I52 is fringed by the inappropriateness marker I57. The appropriateness marker I56 and the inappropriateness marker I57 are drawn in different color values and/or line types so that they can be visually distinguished from each other. For example, it suffices that a thin green line covering the periphery of the corresponding line segment range is displayed as an appropriateness marker I56, and a thick red line is displayed as an inappropriateness marker I57. The markers I56 and I57 may be superimposed on a line indicating the device size.

The marker indicating a determination result regarding appropriateness of the implantation of the left atrial appendage closure device is not limited to a line ornamented with a color value and/or a line type. For example, a character string indicating (in) appropriateness of the implantation of the left atrial appendage closure device may be added, or a color or hatching may be added to the entire or a part of the corresponding line segment angle range.

### Modification 3

The processing circuitry 25 according to Modification 3 may measure a dimension of the left atrial appendage closure device before a tensile test based on a three-dimensional ultrasonic image collected before the tensile test and a dimension of the left atrial appendage closure device after a tensile test based on a three-dimensional ultrasonic image collected after the tensile test. In the three-dimensional ultrasonic image collected before the tensile test, the left atrial appendage closure device implanted in the left atrial appendage before the tensile test is shown; in the three-dimensional ultrasonic image collected after the tensile test, on the other hand, the left atrial appendage closure device implanted in the left atrial appendage after the tensile test is shown. If the adhesion of the left atrial appendage closure device is weak, the left atrial appendage closure device would move to the vicinity of the entrance of the left atrial appendage as a result of a tensile test. The processing circuitry 25 displays a measured value of the dimension before the tensile test of the device and a measured value of the dimension after the tensile test. The processing circuitry 25 causes the display device 29 to display a display screen showing measured values of the dimension before and after the tensile test. It suffices that the measured values of the dimension before and after the tensile test are displayed in a device diameter map or the device diameter graph.

FIG. 14 is a diagram illustrating a device diameter map I6 showing measured values of dimensions before and after a tensile test. As shown in FIG. 14, the measured value I61 before the tensile test, the measured value I62 after the tensile test, the proper values I63, I64, and 165, and the device size I66 are drawn in the device diameter map I6 for each line segment angle around the axis core point I60. The proper range I63, the upper-limit proper value I64, and the lower-limit proper value I65 are displayed as the proper values. According to the device diameter map I6, the user can ascertain a degree of deviation between the measured value I61 before the tensile test and the measured value I62 after the tensile test for each line segment angle. It is also possible to compare the measured values I61 and I62 before and after the tensile test with the proper range I63 and the device size I66 for each line segment angle. It is thus possible for the user to ascertain the implantation state of the left atrial appendage closure device in more detail.

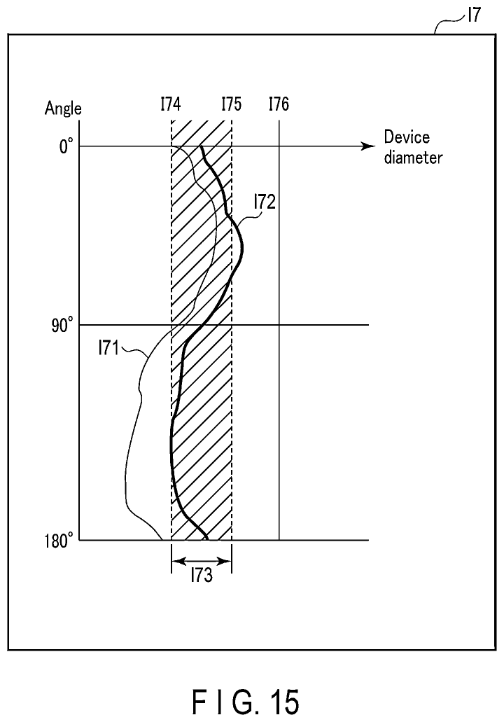

FIG. 15 is a diagram illustrating a device diameter graph I7 showing measured values of dimensions before and after a tensile test. As shown in FIG. 15, the measured value I71 before the tensile test, the measured value I72 after the tensile test, the proper values I73, I74, and I75, and the device size I76 are drawn in the device diameter graph I7 for each line segment angle around the axis core point. The proper range I73, the upper-limit proper value I74, and the lower-limit proper value I75 are displayed as the proper values. It is also possible for the user to ascertain a degree of deviation between, for example, the measured value I71 before the tensile test and the measured value I72 after the tensile test for each line segment angle in the device diameter graph I7, similarly to the device diameter map I6 shown in FIG. 14.

### Modification 4

The storage apparatus 26 according to Modification 4 stores chronological frame data of a three-dimensional ultrasonic image, the frame data being associated with an electrocardiographic time phase. The chronological frame data of the three-dimensional ultrasonic image can be collected by video imaging by three-dimensional ultrasonic scanning. An electrocardiogram of a subject is collected through an electrocardiogram parallel to the three-dimensional ultrasonic scanning, and the electrocardiogram is transferred to the ultrasonic diagnosis apparatus 100 from the electrocardiogram. The storage apparatus 26 stores the frame data and the electrocardiographic time phase, associating collection times thereof with a reference. The processing circuitry 25 specifies, from the electrocardiographic time phase, a maximum time phase at which the dimension of the left atrial appendage closure device becomes a maximum value and a minimum time phase at which the dimension of the left atrial appendage closure device becomes a minimum value. The processing circuitry 25 displays a measured value of the dimension of the left atrial appendage closure device in the maximum time phase and/or a measured value of the dimension of the left atrial appendage closure device in a minimum time phase. It suffices that the measured values are displayed in a device diameter map or a device diameter graph.

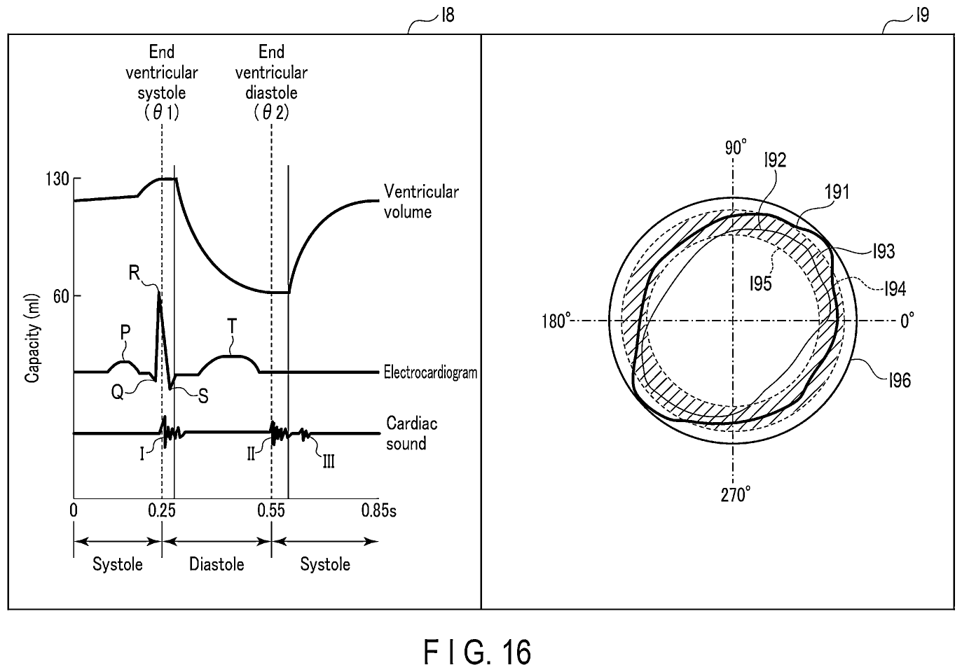

FIG. 16 is a diagram illustrating an electrocardiogram I8 and a device diameter map I9. As shown in FIG. 16, the electrocardiograph I8 shows an electrocardiographic wave of one heartbeat. A maximum time phase at which the dimension of the left atrial appendage closure device takes a maximum value is estimated to be a time phase (end ventricular systole) θ1 immediately after a T wave, at which the ventricular volume takes approximately a minimum value. A minimum time phase at which the dimension of the left atrial appendage closure device takes a minimum value is estimated to be a time phase (end ventricular diastole) θ2 of an R wave, at which the ventricular volume takes approximately a maximum value.

The processing circuitry 25 detects a T wave by analyzing the electrocardiographic waveform in the electrocardiogram I8, specifies the time phase θ1 immediately after the T wave as a maximum time phase, specifies a frame of the three-dimensional ultrasonic image associated with the maximum time phase θ1 (hereinafter, a “maximum time phase frame”), and measures the device diameter of the left atrial appendage closure device for each line segment angle on an axis core orthogonal cross section of the maximum time phase frame. The processing circuitry 25 detects a T wave by analyzing the electrocardiographic waveform in the electrocardiogram I8, specifies the time phase θ1 immediately after the T wave as a maximum time phase, specifies a frame of the three-dimensional ultrasonic image associated with the minimum time phase θ2 (hereinafter, a “minimum time phase frame”), and measures the device diameter of the left atrial appendage closure device for each line segment angle on an axis core orthogonal cross section of the minimum time phase frame.

As shown in FIG. 16, the processing circuitry 25 generates a device diameter map I9 in which at least the measured value I91 of the diameter in the maximum time phase and the measured value I92 of the diameter in the minimum time phase are plotted. The device diameter map I9 is displayed on the display device 29. It is preferable if the proper values I93, I94, and I95 and the device size I96 are additionally drawn in the device diameter map I9. It suffices that the proper range I93, the upper-limit proper value I94, and the lower-limit proper value I95 are displayed as the proper values. With the device diameter map I9, it is possible to ascertain measured values of the device diameter at each line segment angle in a maximum time phase and a minimum time phase, and to compare the measured values with the proper values. It is possible to reduce or prevent overlooking of a time phase at which a measured value does not take a proper value by checking the measured values in multiple time phases.

### Modification 5

The storage apparatus 26 according to Modification 5 stores chronological frame data of a three-dimensional ultrasonic image. The chronological frame data of a three-dimensional ultrasonic image can be collected through video imaging by three-dimensional ultrasonic scanning, which is performed under the control of the transmission/reception control circuitry 23. The processing circuitry 25 measures the dimension of the left atrial appendage closure device for each chronological frame data set, and specifies a maximum frame. The processing circuitry 25 displays a measured value of the dimension of the device in the maximum frame and/or a measured value of the dimension of the device in a minimum frame. The measured value of the dimension of the device in the maximum frame and/or the measured value of the dimension of the device in the minimum frame may be displayed in a device diameter map, similarly to the example of FIG. 16. These measured values may be displayed in a device diameter graph, instead of the device diameter map.

According to Modification 5, it is possible to ascertain measured values of the device diameter at each line segment angle in a maximum frame and a minimum frame, and to compare the measured values with the proper values. It is possible to reduce or prevent overlooking of a frame at which a measured value does not take a proper value by checking the measured values in multiple frames.

### Modification 6

The medical image processing apparatus 2 according to some of the foregoing modifications was an apparatus main body provided in the ultrasonic diagnosis apparatus 100. However, the present embodiment is not limited to this example. The medical image processing apparatus according to Modification 6 is a computer independent from the ultrasonic diagnosis apparatus.

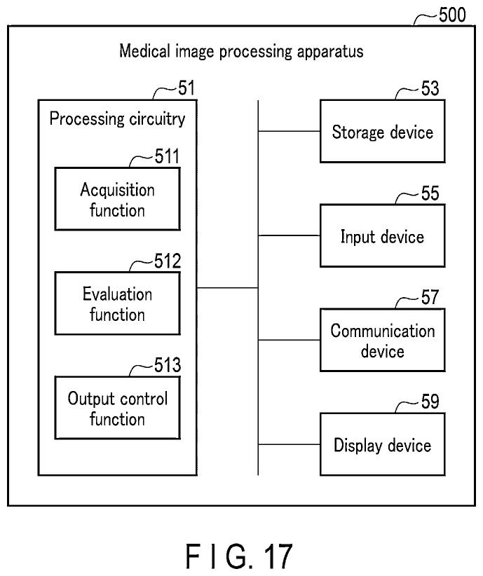

FIG. 17 is a diagram showing a configuration example of a medical image processing apparatus 500 according to Modification 6. As shown in FIG. 17, the medical image processing apparatus 500 is a computer having processing circuitry 51, a storage apparatus 53, an input device 55, a communication device 57, and a display device 59. Data communication between the processing circuitry 51, the storage apparatus 53, the input device 55, the communication device 57, and the display device 59 is performed via a bus.

The processing circuitry 51 has a processor such as a CPU and a CPU that governs the medical image processing apparatus 500. The processing circuitry 51 executes a medical image processing program stored in the storage apparatus 53 to realize a function corresponding to the program. The processing circuitry 51 realizes the acquisition function 511, the evaluation function 512, and the output control function 513, for example. The embodiment is not limited to the case in which the respective functions 511 to 513 are realized by single processing circuitry. Processing circuitry may be composed by combining a plurality of independent processors, and the respective processors may execute programs, thereby realizing the functions 511 to 513. Each of the functions 511 to 513 may be implemented as a module program or as separate hardware.

The acquisition function 511, the evaluation function 512, and the output control function 513 correspond to the acquisition function 252, the evaluation function 253, and the output control function 254, respectively. Suppose that the medical image acquired by the acquisition function 511 is a three-dimensional medical image collected by the medical image diagnosis apparatus. The medical image diagnosis apparatus according to Modification 6 may be another modality other than an ultrasonic diagnosis apparatus, such as an X-ray computer tomography imaging apparatus, an X-ray diagnosis apparatus, a magnetic resonance imaging apparatus, or a nuclear medical diagnosis apparatus.

### Modification 7

The processing circuitry 25 according to the foregoing modifications evaluates an implantation state of the left atrial appendage closure device with respect to a left atrial appendage from multiple angles, using a medical image. However, the present embodiment is not limited to this example. The implantation state of the left atrial appendage closure device with respect to the left atrial appendage may be evaluated for multiple angles using a two-dimensional medical image according to Modification 7. As an example, the processing circuitry 25 may collect a two-dimensional ultrasonic image that crosses the axis core of the left atrial appendage closure device, and measure a diameter of the left atrial appendage closure device for each of multiple line segment angles using the two-dimensional ultrasonic image. The processing circuitry 25 may measure a device diameter of the left atrial appendage closure device at multiple probe angles respectively corresponding to multiple ultrasonic scanning surfaces, using a plurality of axis core parallel cross-sectional images respectively corresponding to the multiple ultrasonic scanning surfaces.

### Review

According to some of the foregoing modifications, the processing circuitry of the medical image processing apparatus acquires a medical image relating to a target part in which a device is implanted. The processing circuitry evaluates an implantation state of the target part of the device from multiple angles, using a medical image. The processing circuitry outputs information based on an evaluation result of the implantation state.

According to the above structures, the implantation state is evaluated from multiple angles, and this allows a user to ascertain the implantation state after accurately ascertaining the shape of the device, and overlooking of a cross section that decisively affects the evaluation of the implantation state is expected to be reduced.

According to at least one of the foregoing embodiments, it is possible to accurately ascertain an implantation state of a device in a target part.

The term “processor” used in the above explanation indicates, for example, circuitry, such as a CPU, a GPU, or an application specific integrated circuit (ASIC), and a programmable logic device (for example, a simple programmable logic device (SPLD), a complex programmable logic device (CPLD), and a field programmable gate array (FPGA)). The processor realizes its function by reading and executing the program stored in the storage circuitry. The program may be directly incorporated into the circuitry of the processor instead of being stored in the storage circuitry. In this case, the processor implements the function by reading and executing the program incorporated into the circuitry. If the processor is for example an ASIC, on the other hand, the function is directly implemented in circuitry of the processor as a logic circuit, instead of storing a program in storage circuitry. Each processor of the present embodiment is not limited to a case where each processor is configured as a single circuit; a plurality of independent circuits may be combined into one processor to realize the function of the processor. Furthermore, a plurality of constituent elements shown in FIGS. 1 and 17 may be integrated into one processor to implement the functions.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A medical image processing apparatus comprising processing circuitry configured to:
acquire a medical image relating to a target part at which a device is implanted;
evaluate an implantation state of the device in relation to the target part from multiple angles using the medical image; and
output information based on an evaluation result of the implantation state.

2. The medical image processing apparatus according to claim 1, wherein
the processing circuitry is configured to:
acquire a three-dimensional medical image acquired by a medical image diagnosis apparatus as the medical image; and
evaluate the implantation state from a plurality of angles around the axis core of the device.

3. The medical image processing apparatus according to claim 2, wherein
the processing circuitry is configured to:
measure a dimension of the device implanted in the target part at each of the angles based on the three-dimensional medical image; and
display, on a display device, the measured value of the dimension at each of the angles.

4. The medical image processing apparatus according to claim 3, wherein
the processing circuitry displays the measured value and proper values of the dimension at each of the angles in a map or a graph.

5. The medical image processing apparatus according to claim 4, wherein
the processing circuitry is configured to:
display a cross-sectional image of a cross section orthogonal to an axis core of the device in the three-dimensional medical image; and
display the measured value and the proper values of the dimension at each of the angles on the cross-sectional image.

6. The medical image processing apparatus according to claim 4, wherein
the processing circuitry is configured to:
determine appropriateness of an implantation of the device at the target part based on the measured value and the proper values of the dimension for each of the angles; and
display a determination result relating to appropriateness of the implantation of the device having the dimension for each of the angles.

7. The medical image processing apparatus according to claim 6, wherein
the processing circuitry is configured to display an appropriate angle range in which the implantation of the device is appropriate and an inappropriate angle range in which the implantation of the device is inappropriate in different colors.

8. The medical image processing apparatus according to claim 3, wherein
the processing circuitry is configured to:
measure the dimension of the device before a tensile test based on the three-dimensional medical image collected before the tensile test of the device and the dimension of the device after a tensile test based on the three-dimensional medical image collected after the tensile test of the device; and
display the measured value of the dimension before the tensile test of the device and the measured value of the dimension after the tensile test.

9. The medical image processing apparatus according to claim 3, further comprising a memory storing chronological frame data of the three-dimensional medical image, the frame data being associated with an electrocardiographic time phase, wherein
the processing circuitry is configured to:
specify, in the electrocardiographic time phase, a maximum time phase at which the dimension of the device becomes a maximum value and/or a minimum time phase at which the dimension of the device becomes a minimum value, and measure the dimension of the device based on the three-dimensional medical image of a frame corresponding to the maximum time phase and/or the dimension of the device based on the three-dimensional medical image of a frame corresponding to the minimum time phase; and
display the measured value of the dimension of the device at the maximum time phase and/or the measured value of the dimension of the device at the minimum time phase.

10. The medical image processing apparatus according to claim 3, further comprising a memory storing chronological frame data of the three-dimensional medical image, wherein
the processing circuitry is configured to:
measure the dimension of the device for each piece of chronological frame data, and specify a maximum frame and/or a minimum frame at which the dimension of the device becomes a maximum value or a minimum value based on the measured value of the dimension of the device of each piece of chronological frame data; and
display the measured value of the dimension of the device in the maximum frame and the measured value of the dimension of the device in the minimum frame.

11. The medical image processing apparatus according to claim 2, wherein
the medical image diagnosis apparatus is an ultrasonic diagnosis apparatus, an X-ray computer tomography imaging apparatus, a magnetic resonance value simulation apparatus, or a nuclear medicine diagnosis apparatus.

12. The medical image processing apparatus according to claim 1, wherein
the target part is a left atrial appendage, and
the device is a left atrial appendage closure device.

13. An ultrasonic image processing method comprising:
acquiring a medical image relating to a target part at which a device is implanted;
evaluating an implantation state of the device in relation to the target part from multiple angles using the medical image; and
outputting information based on an evaluation result of the implantation state.

14. An ultrasonic diagnosis apparatus comprising:
signal processing circuitry configured to generate a medical image relating to a target part in which a device is implanted based on an echo signal from an ultrasonic probe that receives or transmits an ultrasonic wave from and to a subject in which the device is implanted in the target part; and
processing circuitry configured to evaluate an implantation state of the device with respect to the target part from multiple angles using the medical data, and output information based on a result of the evaluation of the implantation state.
